(19) United States
(12) Patent Application Publication
(10) Pub. No.: US 2026/0048240 A1
(43) Pub. Date: Feb. 19, 2026

(54) HANDLE FOR A STEERABLE MEDICAL DEVICE

(71) Applicant: Boston Scientific Scimed, Inc., Maple Grove, MN (US)

(72) Inventor: Jeffery D. ARNETT, Louisa, VA (US)

(21) Appl. No.: 19/271,052

(22) Filed: Jul. 16, 2025

Related U.S. Application Data
(60) Provisional application No. 63/683,118, filed on Aug. 14, 2024.

(51) Int. Cl. A61M 25/09; A61M 25/01

(52) U.S. Cl. CPC A61M 25/09; A61M 25/0136; A61M 2025/09116

(57) ABSTRACT

A handle for a steerable medical device including a plurality of control wires, a distal end of the plurality of control wires being coupled to the medical device at a distal region thereof, includes a grip having a distal end and a proximal end. A spindle extends from the distal end of the grip and includes a channel for receiving the control wires and a plurality of openings through which the plurality of control wires extend. A first knob is rotatably coupled to the spindle. The first knob is configured for actuating a first wire holder to manipulate a first control wire to produce a change in deflection of the medical device in a first direction and actuating a second wire holder to manipulate a second control wire to produce a change in deflection of the medical device in a second direction. The handle includes a rotatable lock.

### CROSS REFERENCE TO RELATED APPLICATIONS

This application claims priority to U.S. Provisional Patent Application No. 63/683,118 entitled “HANDLE FOR A STEERABLE MEDICAL DEVICE,” filed Aug. 14, 2024, which is hereby incorporated by reference in its entirety.

### TECHNICAL FIELD

The disclosure relates to a handle for a medical device. More specifically the disclosure relates to a handle for a medical device that enables steering of the medical device in a body of a patient.

### BACKGROUND

U.S. Pat. No. 5,944,690 granted to Falwell et al. discloses a steerable catheter control mechanism for manipulating a pair of control wires which utilizes a slider mechanism coupled to the proximal ends of the control wires. However, the slider mechanism disclosed by Falwell lacks ease of use as it is awkward to grasp and use. Furthermore, the disclosed slider mechanism provides limited control in steering the catheter. The device provides a thumb control that lacks precision. It is unable to provide precise steering of the catheter as it lacks resolution for permitting minute manipulations needed to provide slight changes in the deflection of the catheter.

U.S. Pat. No. 7,691,095 granted to Bednarek et al. discloses a bi-directional steerable catheter control handle which includes an adjustment knob rotatably connected to the handle. Rotation of the handle results in deflection of two sliding members (each connected to a pull wire) in opposite directions, resulting in respective deflection of the distal end of the catheter. However, the steerable control handle provided by Bednarek is complex and difficult to manufacture.

### SUMMARY

Example 1 is a handle for a steerable medical device including a plurality of control wires, a distal end of the plurality of control wires being coupled to the medical device at a distal region thereof. The handle includes a grip having a distal end and a proximal end. A spindle extends from the distal end of the grip. The spindle includes a channel for receiving the plurality of control wires and a plurality of openings through which a proximal portion of the plurality of control wires extend. A first knob is rotatably coupled to the spindle. The first knob is configured for actuating a first wire holder to manipulate a first control wire of the plurality of control wires to produce a change in a deflection of the medical device in a first direction. The first knob is also configured for actuating a second wire holder to manipulate a second control wire of the plurality of control wires to produce a change in a deflection of the medical device in a second direction opposite the first direction. The handle includes a rotatable lock for locking the first knob to maintain a desired shape of the medical device.

Example 2 is the handle of Example 1, further comprising a second knob rotatably coupled to the spindle. The second knob is configured for actuating a third wire holder to manipulate a third control wire of the plurality of control wires to produce a change in a deflection of the medical device in a third direction. The second knob is also configured for actuating a fourth wire holder to manipulate a fourth control wire of the plurality of control wires to produce a change in a deflection of the medical device in a fourth direction opposite the third direction.

Example 3 is the handle of Examples 1 or 2, wherein the spindle further includes a threaded distal end and a longitudinal groove opening to the channel.

Example 4 is the handle of Example 3, wherein the spindle includes a pair of ribs extending along the longitudinal groove.

Example 5 is the handle of Examples 3 or 4, further comprising at least one anti-rotation washer, wherein the anti-rotation washer includes a tang configured to be received in the longitudinal groove.

Example 6 is the handle of Example 5, wherein the at least one anti-rotation washer includes a first anti-rotation washer positioned between the first knob and the second knob and a second anti-rotation washer positioned between the second knob and the rotatable lock.

Example 7 is the handle of any of Examples 2-6, wherein the rotatable lock includes an inner surface having a plurality of ramps that interact with a cam washer having a plurality of ramps, and wherein rotation of the rotatable lock causes a compressive force that locks the first knob and the second knob to maintain a desired shape of the medical device.

Example 8 is the handle of any of Examples 3-7, further comprising a nose cap including internal threads configured for mating with the threaded distal end of the spindle.

Example 9 is the handle of any of Examples 2-8, wherein the first knob includes an internal surface having a first raised portion and a second raised portion extending parallel to a longitudinal axis of the handle, and the second knob includes an internal surface having a third raised portion and a fourth raised portion extending parallel to the longitudinal axis of the handle.

Example 10 is the handle of any of Examples 2-9, wherein the first wire holder, second wire holder, third wire holder, and fourth wire holder include a first portion having a first diameter and a second portion having a second diameter.

Example 11 is the handle of Example 10, wherein the first wire holder, second wire holder, third wire holder, and fourth wire holder include a first internal shoulder and second internal shoulder located on an inner surface adjacent junctions between the first portion and the second portion.

Example 12 is the handle of Examples 10 or 11, wherein the first wire holder, second wire holder, third wire holder, and fourth wire holder include an outer shoulder located on an outer surface adjacent a junction between the first portion and the second portion.

Example 13 is the handle of Example 12, wherein the outer shoulder of the first wire holder is configured to engage with the first raised portion when the first knob is rotated in a first direction and the outer shoulder of the second wire holder is configured to engage with the second raised portion when the first knob is rotated in a second direction.

Example 14 is the handle of Example 13, wherein the outer shoulder of the third wire holder is configured to engage with the third raised portion when the second knob is rotated in a first direction and the outer shoulder of the fourth wire holder is configured to engage with the fourth raised portion when the second knob is rotated in a second direction.

Example 15 is the handle of Example 14, wherein the first internal shoulder and the second internal shoulder of the first wire holder, second wire holder, third wire holder, or fourth wire holder are configured to contact the pair of ribs to limit rotation of the first wire holder, second wire holder, third wire holder, and fourth wire holder, respectively, between a neutral position and a maximum deflection position.

Example 16 is a handle for a steerable medical device including a plurality of control wires, a distal end of the plurality of control wires being coupled to the medical device at a distal region thereof. The handle includes a grip having a distal end and a proximal end. A spindle extends from the distal end of the grip. The spindle includes a channel for receiving the plurality of control wires and a plurality of openings through which a proximal portion of the plurality of control wires extend. A first knob is rotatably coupled to the spindle. The first knob is configured for actuating a first wire holder to manipulate a first control wire of the plurality of control wires to produce a change in a deflection of the medical device in a first direction. The first knob is also configured for actuating a second wire holder to manipulate a second control wire of the plurality of control wires to produce a change in a deflection of the medical device in a second direction opposite the first direction. A second knob is rotatably coupled to the spindle. The second knob is configured for actuating a third wire holder to manipulate a third control wire of the plurality of control wires to produce a change in a deflection of the medical device in a third direction. The second knob is also configured for actuating a fourth wire holder to manipulate a fourth control wire of the plurality of control wires to produce a change in a deflection of the medical device in a fourth direction opposite the third direction. The handle includes a rotatable lock for locking the first knob and the second knob to maintain a desired shape of the medical device.

Example 17 is the handle of Example 16, wherein the spindle further includes a threaded distal end and a longitudinal groove opening to the channel.

Example 18 is the handle of Example 17, wherein the spindle includes a pair of ribs extending along the longitudinal groove.

Example 19 is the handle of Example 17, further comprising at least one anti-rotation washer, wherein the anti-rotation washer includes a tang configured to be received in the longitudinal groove.

Example 20 is the handle of Example 19, wherein the at least one anti-rotation washer includes a first anti-rotation washer positioned between the first knob and the second knob and a second anti-rotation washer positioned between the second knob and the rotatable lock.

Example 21 is the handle of Example 16, wherein the rotatable lock includes an inner surface having a plurality of ramps that interact with a cam washer having a plurality of ramps, and wherein rotation of the rotatable lock causes a compressive force that locks the first knob and the second knob.

Example 22 is the handle of Example 17, further comprising a nose cap including internal threads configured for mating with the threaded distal end of the spindle.

Example 23 is the handle of Example 16, wherein the first knob includes an internal surface having a first raised portion and a second raised portion extending parallel to a longitudinal axis of the handle, and the second knob includes an internal surface having a third raised portion and a fourth raised portion extending parallel to the longitudinal axis of the handle.

Example 24 is the handle of Example 18, wherein the first wire holder, second wire holder, third wire holder, and fourth wire holder include a first portion having a first diameter and a second portion having a second diameter.

Example 25 is the handle of Example 24, wherein the first wire holder, second wire holder, third wire holder, and fourth wire holder include a first internal shoulder and second internal shoulder located on an inner surface adjacent junctions between the first portion and the second portion.

Example 26 is the handle of Example 25, wherein the first wire holder, second wire holder, third wire holder, and fourth wire holder include an outer shoulder located on an outer surface adjacent a junction between the first portion and the second portion.

Example 27 is the handle of Example 26, wherein the outer shoulder of the first wire holder is configured to engage with the first raised portion when the first knob is rotated in a first direction and the outer shoulder of the second wire holder is configured to engage with the second raised portion when the first knob is rotated in a second direction.

Example 28 is the handle of Example 27, wherein the outer shoulder of the third wire holder is configured to engage with the third raised portion when the second knob is rotated in a first direction and the outer shoulder of the fourth wire holder is configured to engage with the fourth raised portion when the second knob is rotated in a second direction.

Example 29 is the handle of claim 25, wherein the first internal shoulder and the second internal shoulder of the first wire holder, second wire holder, third wire holder, or fourth wire holder are configured to contact the pair of ribs to limit rotation of the first wire holder, second wire holder, third wire holder, and fourth wire holder, respectively, between a neutral position and a maximum deflection position.

Example 30 is a handle for a steerable medical device including a plurality of control wires, a distal end of the plurality of control wires being coupled to the medical device at a distal region thereof. The handle includes a grip having a distal end and a proximal end. A spindle extends from the distal end of the grip. The spindle includes a threaded distal end, a channel for receiving the plurality of control wires, and a plurality of openings through which a proximal portion of the plurality of control wires extend. A first knob is rotatably coupled to the spindle. The first knob is configured for actuating a first wire holder to manipulate a first control wire of the plurality of control wires to produce a change in a deflection of the medical device in a first direction. The first knob is also configured for actuating a second wire holder to manipulate a second control wire of the plurality of control wires to produce a change in a deflection of the medical device in a second direction opposite the first direction. The handle includes a rotatable lock for locking the first knob to maintain a desired shape of the medical device. The handle includes a nose cap having internal threads configured for mating with the threaded distal end of the spindle.

Example 31 is the handle of Example 30, further comprising a second knob rotatably coupled to the spindle, for actuating a third wire holder to manipulate a third control wire of the plurality of control wires to produce a change in a deflection of the medical device in a third direction and for actuating a fourth wire holder to manipulate a fourth control wire of the plurality of control wires to produce a change in a deflection of the medical device in a fourth direction opposite the third direction.

Example 32 is the handle of Example 30, wherein the nose cap is configured to retain a proximal end of a tubular member in the channel.

Example 33 is a handle for a steerable medical device including a plurality of control wires, a distal end of the plurality of control wires being coupled to the medical device at a distal region thereof. The handle includes a grip having a distal end and a proximal end. A spindle extends from the distal end of the grip. The spindle includes a channel for receiving the plurality of control wires and a plurality of openings through which a proximal portion of the plurality of control wires extend. Each of the plurality of openings includes a plurality of curved surfaces. A first knob is rotatably coupled to the spindle. The first knob is configured for actuating a first wire holder to manipulate a first control wire of the plurality of control wires to produce a change in a deflection of the medical device in a first direction. The first knob is also configured for actuating a second wire holder to manipulate a second control wire of the plurality of control wires to produce a change in a deflection of the medical device in a second direction opposite the first direction. A second knob is rotatably coupled to the spindle. The second knob is configured for actuating a third wire holder to manipulate a third control wire of the plurality of control wires to produce a change in a deflection of the medical device in a third direction. The second knob is also configured for actuating a fourth wire holder to manipulate a fourth control wire of the plurality of control wires to produce a change in a deflection of the medical device in a fourth direction opposite the third direction. The handle includes a rotatable lock for locking the first knob and the second knob to maintain a desired shape of the medical device.

Example 34 is the handle of Example 33, wherein the plurality of curved surfaces includes a first curved surface and a second curved surface extending in a plane orthogonal to a longitudinal axis of the handle, and a third curved surface extending in a plane parallel to the longitudinal axis of the handle.

Example 35 is the handle of Example 34, wherein the first curved surface and the second curved surface are configured such that the plurality of openings are larger at an exterior surface of the spindle and smaller at the channel.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a handle assembly, in accordance with an embodiment the present disclosure.

FIG. 2 in an exploded view of the handle assembly of FIG. 1, in accordance with an embodiment of the present disclosure.

FIG. 3 is a perspective view of a spindle of the handle assembly of FIG. 1, in accordance with an embodiment of the present disclosure.

FIG. 4 is a perspective view of wire holders being positioned on a spindle of the handle assembly of FIG. 1, in accordance with an embodiment of the present disclosure.

FIG. 5 is a perspective view of a proximal knob being positioned over a first and second wire holder on a spindle of the handle assembly of FIG. 1, in accordance with an embodiment of the present disclosure.

FIG. 6 is a perspective view of an anti-rotation washer being positioned over a spindle of the handle assembly of FIG. 1, in accordance with an embodiment of the present disclosure.

FIG. 7 is a perspective view of a third wire holder, fourth wire holder, distal knob, and second anti-rotation washer being positioned over a spindle of the handle assembly of FIG. 1, in accordance with an embodiment of the present disclosure.

FIG. 8 is a perspective view showing a lock knob and a cam washer being positioned over a spindle of the handle assembly of FIG. 1, in accordance with an embodiment of the present disclosure.

FIG. 9 is a perspective view showing a nose cap being positioned over a distal end of a spindle of the handle assembly of FIG. 1, in accordance with an embodiment of the present disclosure.

FIGS. 10A-10C are cross-sectional views of the handle assembly of FIG. 1 along line 10-10 of FIG. 6 showing actuation of a proximal knob and corresponding operation of the first and second wire holder, in accordance with an embodiment of the present disclosure.

FIG. 11 is a perspective view of a locking knob and a cam washer of the handle assembly of FIG. 1, in accordance with an embodiment of the present disclosure.

FIG. 12 is a cross-sectional view of a spindle of the handle assembly along line 12-12 of FIG. 1, in accordance with an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Steerable medical devices have various uses and applications, such as for guiding and positioning devices such as catheters, dilators, guidewires, sheaths and the like within a patient's body. Handles used with such steerable devices typically include a mechanism for actuating one or more pull wires capable of deflecting the steerable device and thus steering or guiding a functional tip of a medical device positioned therein.

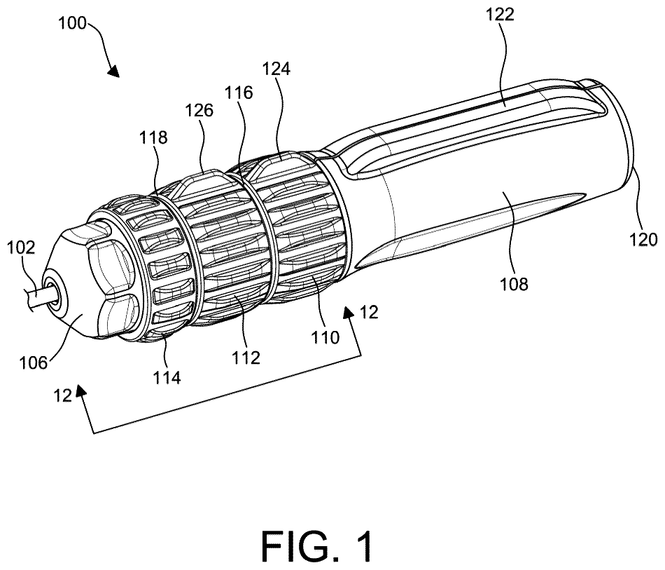

In one embodiment of the present disclosure as shown in FIG. 1, a steerable control assembly or handle 100 is provided for manipulating a medical device 102. The medical device 102 may include, without limitation, a catheter, sheath, dilator, introducer, guidewire, stylet, or similar medical devices. The medical device 102 includes a plurality of control wires for producing a change in deflection of the medical device 102 to allow the medical device 102 to achieve a desired shape. A distal end of the plurality of control wires are coupled to the medical device 102 at a distal region thereof such that a tension applied to a proximal end results in a bend in the medical device 102. The plurality of control wires extend proximally from the connection through one or more channels or lumens to the handle 100. In some embodiments, the medical device 102 may take the form of a tubular member with one or more lumens. In other embodiments, the medical device may include a substantially solid cross-section except for any lumens or grooves configured for holding one or more control wire.

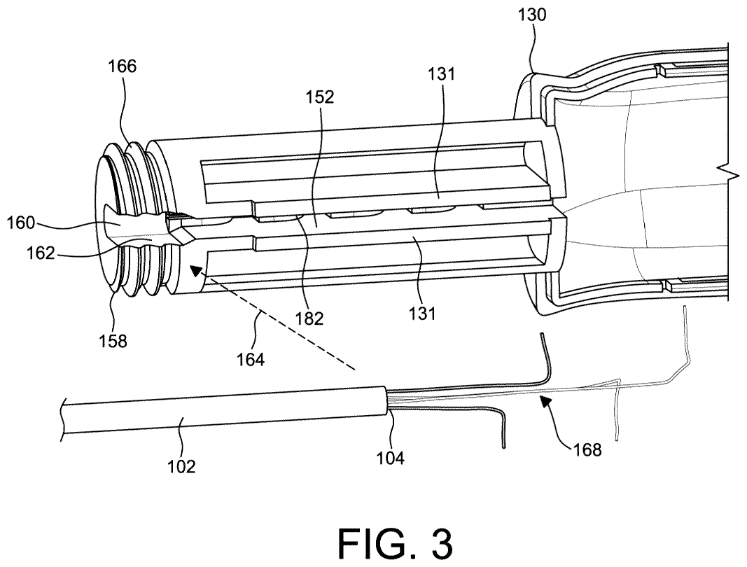

The handle 100 is coupled to the medical device 102 to enable a user to change the shape of the medical device 102 to manipulate or steer the medical device 102 in a desired direction during use. The medical device 102 includes a proximal end 104 (shown in FIG. 3) that is removably affixed within the handle 100 by a nose cap 106.

As illustrated in FIG. 1, the handle 100 include a grip 108, a proximal knob 110, a distal knob 112, a rotatable lock knob 114, nose cap 106, proximal anti-rotation washer 116, and distal anti-rotation washer 118. The grip 108 includes a proximal end 120 and a raised feature 122. The raised feature 122 provides a reference for the medical device 102 in a neutral, or non-deflected orientation. The proximal knob 110 includes a first position indicator 124 and the distal knob 112 includes a second position indicator 126. The proximal knob 110 and the distal knob 112 are configured to be rotated in both a clockwise and counterclockwise direction to change a shape of the medical device 102. When the position indicators 124, 126 are aligned with the raised feature 122, the medical device 102 is substantially straight, or in a neutral position.

Rotating the proximal knob 110 in a clockwise direction puts tension on a control wire and causes the medical device 102 to deflect in a first direction. Rotating the proximal knob 110 in a counterclockwise direction puts tension on a different control wire and causes the medical device to deflect in a second direction that is substantially opposite the first direction. Similarly, rotating the distal knob 112 in a clockwise direction puts tension on another control wire and causes the medical device 102 to deflect in a third direction that is substantially orthogonal to the first and second directions. Rotating the proximal knob 112 in a counterclockwise direction puts tension on an additional control wire and causes the medical device to deflect in a fourth direction that is substantially opposite the third direction. By adjusting both the proximal knob 110 and the distal knob 112 simultaneously, the medical device 102 is able to achieve deflection in any direction. By comparing the locations of the position indicators 124, 126 to the raised feature 122, a user can visualize a shape of the medical device 102 located in a patient.

The rotatable lock knob 114 is configured to selectively inhibit rotation of the proximal knob 110 and the distal knob 112 such that a desired shape of the medical device 102 can be maintained. As used herein, the term lock refers to a structure configured to create a friction force sufficient to inhibit rotation of the knobs, such that the knobs maintain their position when not manipulated by a user. During use, however, the user can readily apply sufficient torque to overcome the friction force and cause the knobs to rotate.

A user can rotate the proximal knob 110 and/or the distal knob 112 to deflect the medical device 102 as desired, then actuate the rotatable lock knob 114 to lock the proximal knob 110 and the distal knob 112 in place. The rotatable lock knob 114 provides a force towards the proximal end 120 of the grip 108 that compress the proximal knob 110, proximal anti-rotation washer 116, distal knob 112, and distal anti-rotation washer 118. This compressive force prevents rotation of proximal knob 110 and distal knob 112. In some aspects, a user may rotate the lock knob 114 to apply a compressive force prior to rotating the proximal knob 110 or the distal knob 112. In this situation, the positioning of the proximal knob 110 or the distal knob 112 will automatically be retained after a user rotates the knobs 110, 112 to a desired position.

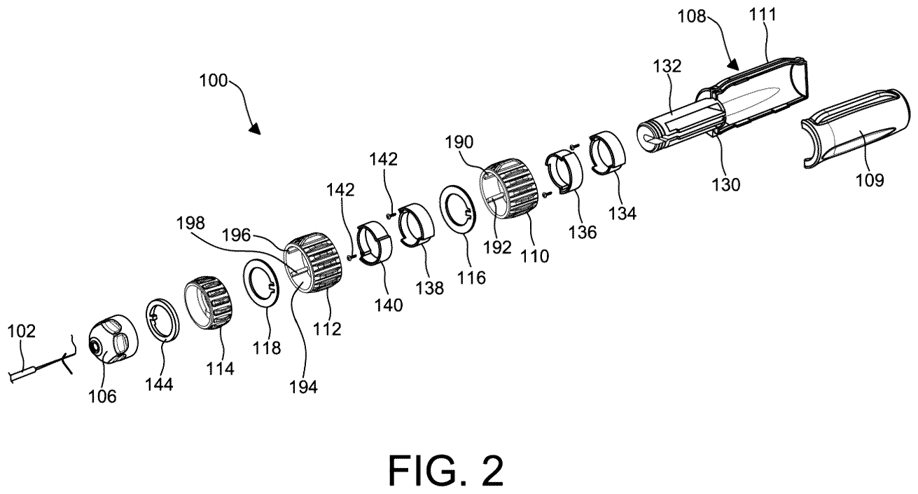

FIG. 2 in an exploded view of the handle assembly of FIG. 1, in accordance with an embodiment of the present disclosure. The grip 108 is formed by two halves joined together by one or more connectors 128, such as clips or snap fit connectors. A left grip portion 109 joins with a right grip portion 111 to form the grip 108. The grip 108 is substantially hollow and includes a proximal end 120 and a distal end 130. In embodiments where the medical device 102 includes one or more electrode or sensor, the grip 108 may house electrical connectors, leads, or other components. A spindle 132 extends from the distal end 130 of the grip. The spindle 132 acts as a support for components of the handle 100.

The handle 100 includes a first wire holder 134 that is configured to attach to a proximal end of first control wire, a second wire holder 136 that is configured to attach to a proximal end of a second control wire, a third wire holder 138 that is configured to attach to a proximal end of a third control wire, and a fourth wire holder 140 that is configured to attach to a proximal end of a fourth control wire. Fasteners 142, such as screws, crimped ferrules, pins, or staples are used to join the proximal end of each of the control wires to the respective wire holder. In some embodiments, the proximal end of the control wires may be secured using an adhesive.

The first wire holder 134 and the second wire holder 136 are actuatable by rotation of the proximal knob 110. The proximal knob 110 only engages a single wire holder during rotation. When the proximal knob 110 is rotated in a first direction, for example clockwise, the first wire holder 134 is actuated to manipulate a first control wire to produce a change in a deflection of the medical device 102 in a first direction. When the proximal knob 110 is rotated in a second direction, for example counterclockwise, the second wire holder 136 is actuated to manipulate a second control wire to produce a change in a deflection of the medical device 102 in a second direction substantially opposite the first direction.

The third wire holder 138 and the fourth wire holder 140 are actuatable by rotation of the distal knob 112. The distal knob 112 only engages a single wire holder during rotation. When the distal knob 112 is rotated in a first direction, for example clockwise, the third wire holder 138 is actuated to manipulate a third control wire to produce a change in a deflection of the medical device 102 in a third direction. When the distal knob 112 is rotated in a second direction, for example counterclockwise, the fourth wire holder 140 is actuated to manipulate a fourth control wire to produce a change in a deflection of the medical device 102 in a fourth direction substantially opposite the third direction.

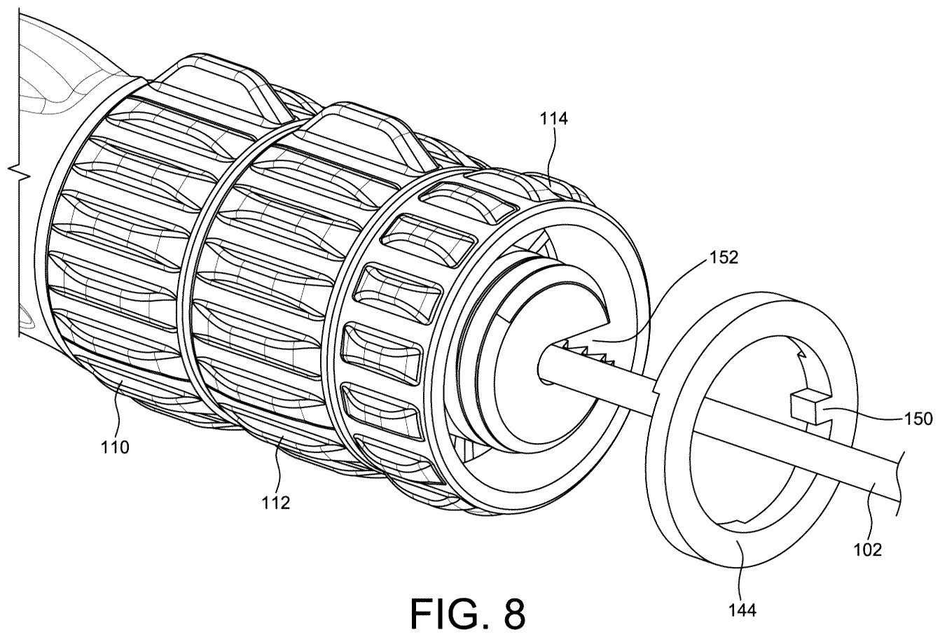

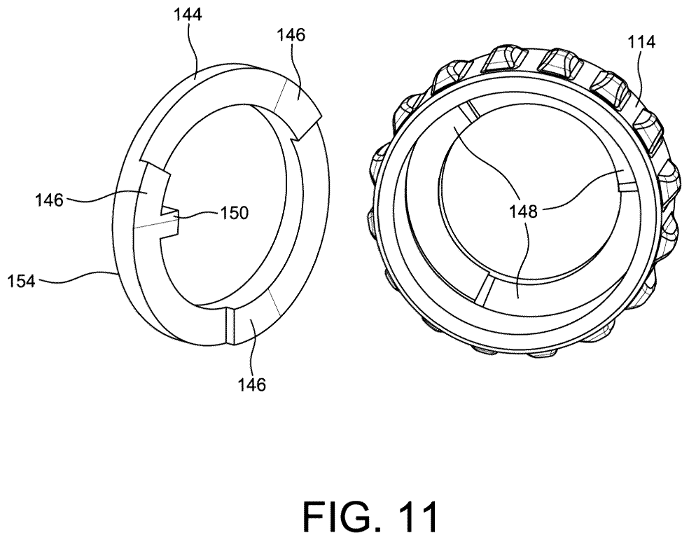

To provide the compressive force to lock the proximal knob 110 and the distal knob 112 at a desired location, the rotatable lock knob 114 interacts with a cam washer 144. As illustrated in FIG. 11, the cam washer 144 includes a plurality of ramped surfaces 146 that interact with corresponding ramped surfaces 148 located on an inner surface of the rotatable lock knob 114. The cam washer 144 includes a tang 150 that rests within a longitudinal groove 152 of spindle 132 to prevent rotation of the cam washer 144 while allowing longitudinal movement. FIG. 8 is a perspective view showing the cam washer 144 being positioned over the spindle 132 so the tang 150 aligns with longitudinal groove 152. As the rotatable lock knob 114 is turned, the ramped surfaces 148 of the knob 114 interact with the ramped surfaces 146 of the cam washer 144 forcing a flat surface 154 of the cam washer 144 against the nose cap 106. Because the nose cap 106 is stationary, a force is generated towards the grip 108 which holds the proximal knob 110 and the distal knob 112 stationary.

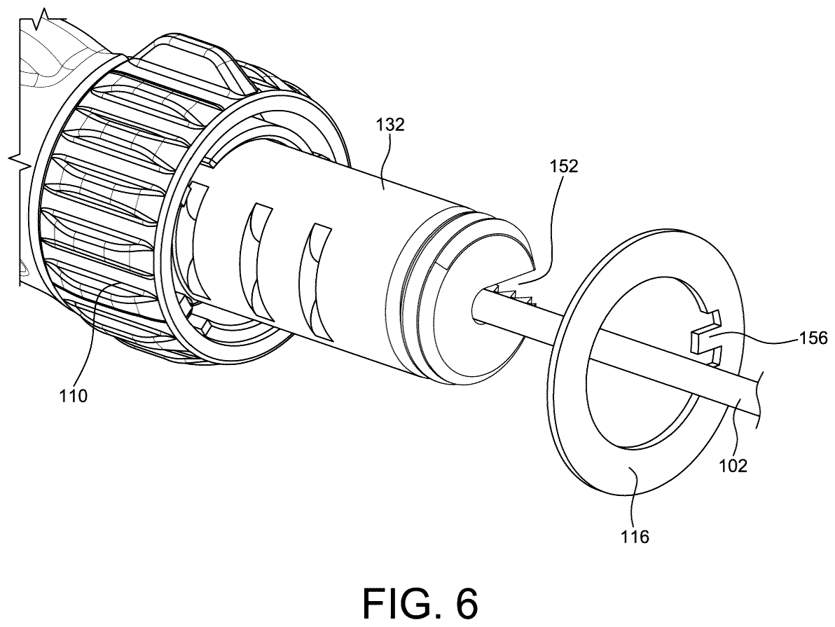

A proximal anti-rotation washer 116 is located between the proximal knob 110 and the distal knob 112 and a distal anti-rotation washer 118 is located between the distal knob 112 and the rotatable lock knob 114. The anti-rotation washers 116, 118 isolate the knobs so that rotation of one knob does not affect the other. As illustrated in FIG. 6, the anti-rotation washers 116, 118 include a tang 156 that rests within a longitudinal groove 152 of spindle 132 to prevent rotation of the anti-rotation washers 116, 118 while allowing longitudinal movement during locking and unlocking of proximal knob 110 and distal knob 112.

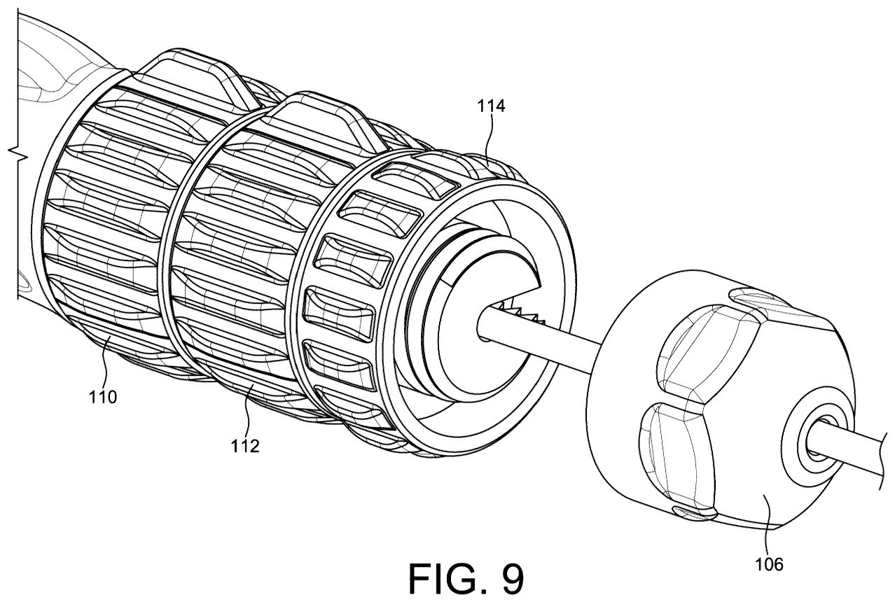

FIG. 3 is a perspective view of the spindle 132 of the handle assembly 100 of FIG. 1, in accordance with an embodiment of the present disclosure. As shown more clearly in FIG. 3, the spindle 132 includes a longitudinal groove 152 that extends from a distal end 158 to the grip distal end 130. A pair of ribs 131 extend along a portion of the longitudinal groove 152. The spindle includes a channel 160 for receiving the plurality of control wires 168. The channel 160 includes a seat 162 configured to receive a proximal end 104 of the medical device 102 as illustrated by arrow 164. Distal end 158 of the spindle 132 includes a threaded portion 166 configured to mate with internal threads of nose cap 106 to retain the proximal end 104 of the medical device 102 in the seat 162. FIG. 9 is a perspective view showing the nose cap 106 being positioned over a distal end of the spindle 132 securing the medical device 102.

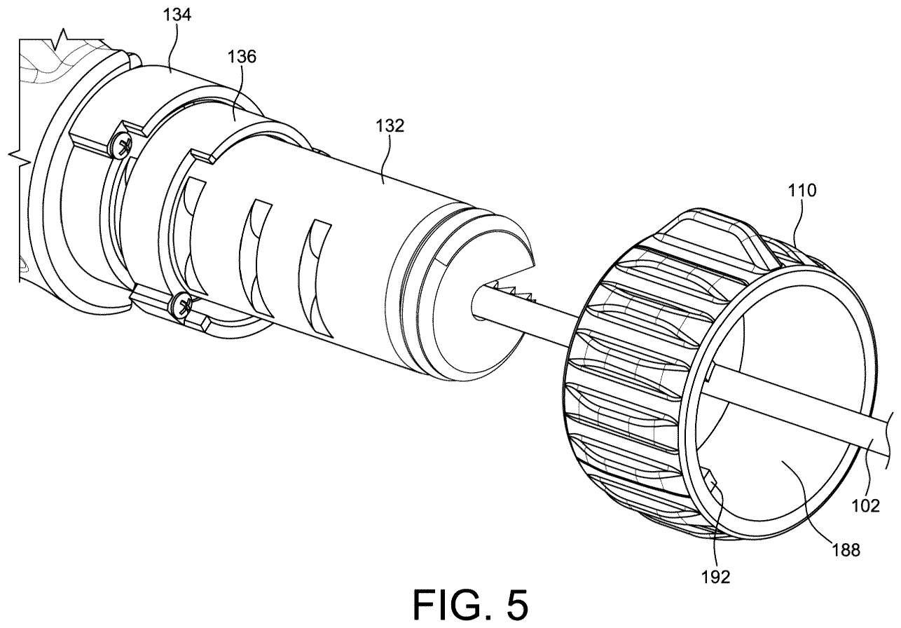

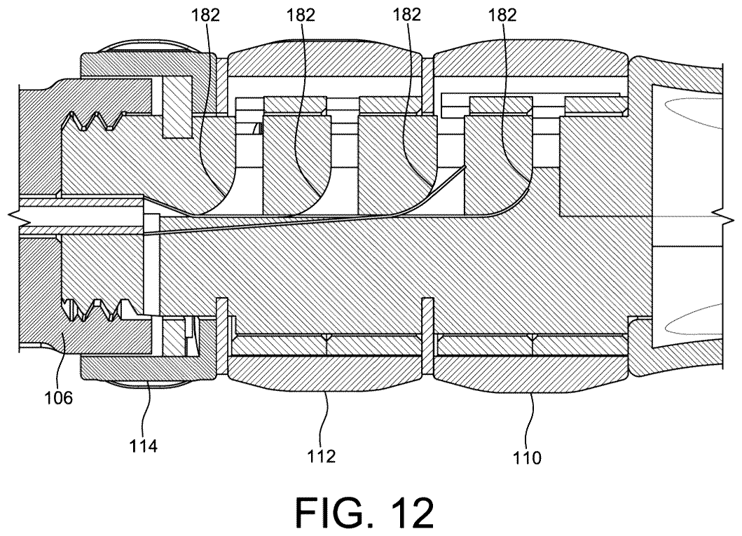

The plurality of control wires 168 extend from the proximal end 104 of the medical device 102, through the channel 160, and exit one of a plurality of openings 170, 172, 174, 176 to be connected to a corresponding wire holder. As illustrated in FIG. 5, the plurality of openings 170, 172, 174, 176 through which a proximal portion of the plurality of control wires 168 extend, each include a plurality of curved surfaces to allow for smooth routing of the control wires 168. Each of the plurality of openings 170, 172, 174, 176 include a first curved surface 178 and a second curved surface 180 extending in a plane orthogonal to a longitudinal axis of the handle 100, the longitudinal axis passing through a center of the spindle 132. The first curved surface 178 and the second curved surface 180 are configured such that the plurality of openings 170, 172, 174, 176 are larger at an exterior surface of the spindle 132 and smaller at the channel 160 at the center of the spindle 132. In other words, the plurality of openings 170, 172, 174, 176 have a funnel shape. The plurality of openings 170, 172, 174, 176 include a third curved surface 182, best viewed in FIGS. 3 and 12, extending in a plane parallel to the longitudinal axis of the handle 100. FIG. 12 is a cross-sectional view of the spindle 132 showing how the plurality of control wires contact the third curved surface 182 to prevent kinks in the wires.

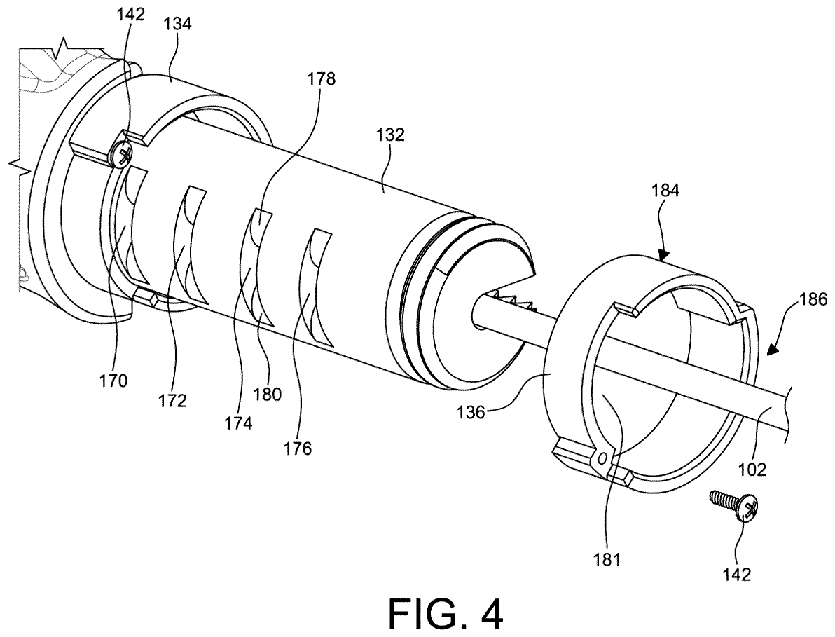

FIG. 4 is a perspective view of the first wire holder 134 on the spindle 132 of the handle assembly of FIG. 1 and the second wire holder 136 being positioned on the spindle 132, in accordance with an embodiment of the present disclosure. In FIG. 4, a proximal end of a first of the plurality of control wires 168 extends out of opening 170 and is attached to the first wire holder 134 using a fastening device 142, such as screw, crimped ferrule, pin, or staple. Upon placing the second wire holder 136 onto spindle 132, a proximal end of a second of the plurality of control wires 168 will extend through opening 172 and be attached to second wire holder 136 using a fastening device 142. Third wire holder 138 and fourth wire holder 140 would similarly be positioned on spindle 132 and joined with a third and fourth control wire respectively.

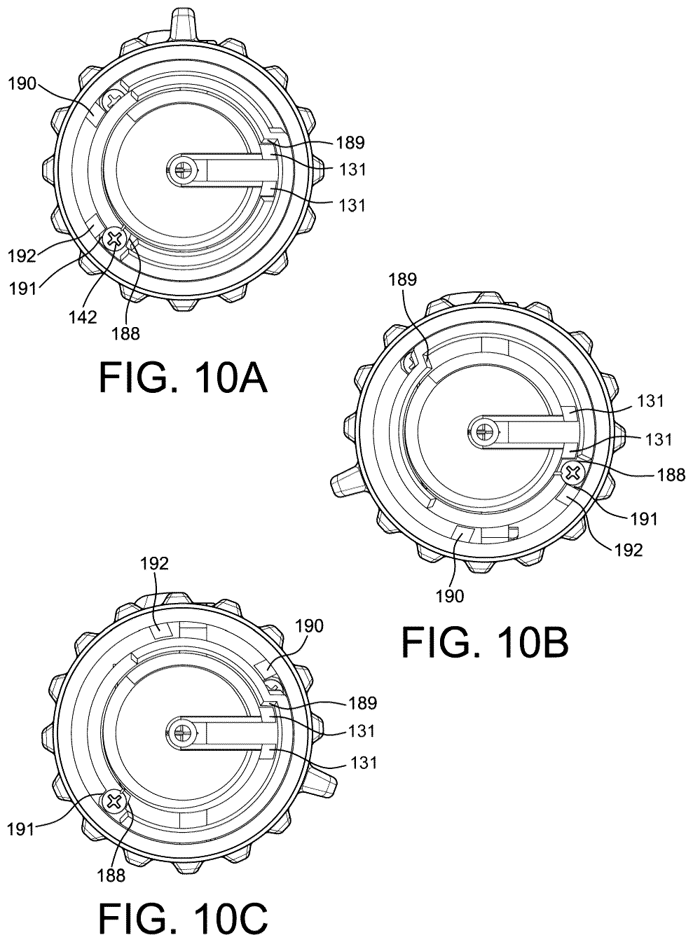

Each of the first wire holder 134, the second wire holder 136, the third wire holder 138, and the fourth wire holder 140 include a generally cylindrical body having a first portion 184 with a first diameter and a second portion 186 with a second diameter that is larger than the first diameter. A cutout 181 allows for rotation of the wire holders 134, 136, 138, 140 without covering a respective opening 170, 172, 174, 176 or a control wire extending therethrough. As best viewed in FIGS. 10A-10C, a first internal shoulder 188 and second internal shoulder 189 are located on an inner surface of each of the wire holders 134, 136, 138, 140 adjacent junctions between the first portion 184 and the second portion 186. The first internal shoulder 188 is adjacent the junction including the fastening device 142, and the second internal shoulder 189 is at the junction opposite the fastening device 142. An outer shoulder 191 is located on an outer surface adjacent a junction between the first portion and the second portion including the fastening device 142. First internal shoulder 188 and second internal shoulder 189 are configured to limit rotation of a wire holder by contacting the pair of ribs 131 that extend along a portion of the longitudinal groove 152. Outer shoulder 191 is configured to contact a portion of the proximal knob 110 or the distal knob 112 as discussed below.

First wire holder 134 and third wire holder 138 are substantially identical. Second wire holder 136 and fourth wire holder 140 are also substantially identical but are mirror images of first wire holder 134 and third wire holder 138.

FIG. 5 is a perspective view of the proximal knob 110 being positioned over the first wire holder 134 and the second wire holder 136, in accordance with an embodiment of the present disclosure. The proximal knob 110 includes an internal surface 188 having a first raised portion 190 and a second raised portion 192. The first raised portion 190 and the second raised portion 192 are spaced apart from one another and extend parallel to the longitudinal axis of the handle in the form of ribs or protrusions. The first raised portion 190 is configured to interact with the outer shoulder 191 of the first wire holder 134 and the second raised portion 192 is configured to interact with the outer shoulder 191 of the second wire holder 136.

Similarly, as shown in FIG. 2, the distal knob 112 includes an internal surface 194 having a third raised portion 196 and a fourth raised portion 198. The third raised portion 196 and the fourth raised portion 198 are spaced apart from one another and extend parallel to the longitudinal axis of the handle in the form of ribs or protrusions. The third raised portion 196 is configured to interact with the outer shoulder 191 of the third wire holder 138 and the fourth raised portion 198 is configured to interact with the outer shoulder 191 of the fourth wire holder 140.

FIGS. 10A-10C are cross-sectional views of the handle assembly of FIG. 1 along line 10-10 of FIG. 6 showing actuation of the proximal knob 110 and corresponding operation of the first wire holder 134 and second wire holder 136, in accordance with an embodiment of the present disclosure. FIG. 10A illustrates the proximal knob 110 in a neutral position where no tension is placed on any of the control wires. In the neutral position, the second internal shoulder 189 of the first wire holder 134 and the second wire holder 136 are in contact with the pair of ribs 131 extending along a portion of the longitudinal groove 152. FIG. 10B illustrates the proximal knob 110 rotated to a maximum counterclockwise position. In this configuration, the first wire holder 134 is unmoved and remains in the neutral position. However, when the proximal knob is rotated counterclockwise, the outer shoulder 191 of the second wire holder 136 is pushed by the second raised portion 192 until the first internal shoulder 188 of the second wire holder 136 contacts the one of the ribs 131. Returning the proximal knob 110 to the neutral position allows the second wire holder 136 to return to the neutral position. FIG. 10C illustrates the proximal knob 110 rotated to a maximum clockwise position. In this configuration, the second wire holder 136 is unmoved and remains in the neutral position. However, when the proximal knob is rotated clockwise, the outer shoulder 191 of the first wire holder 134 is pushed by the first raised portion 190 until the first internal shoulder 188 of the first wire holder 134 contacts one of the ribs 131. Returning the proximal knob 110 to the neutral position allows the first wire holder 134 to return to the neutral position.

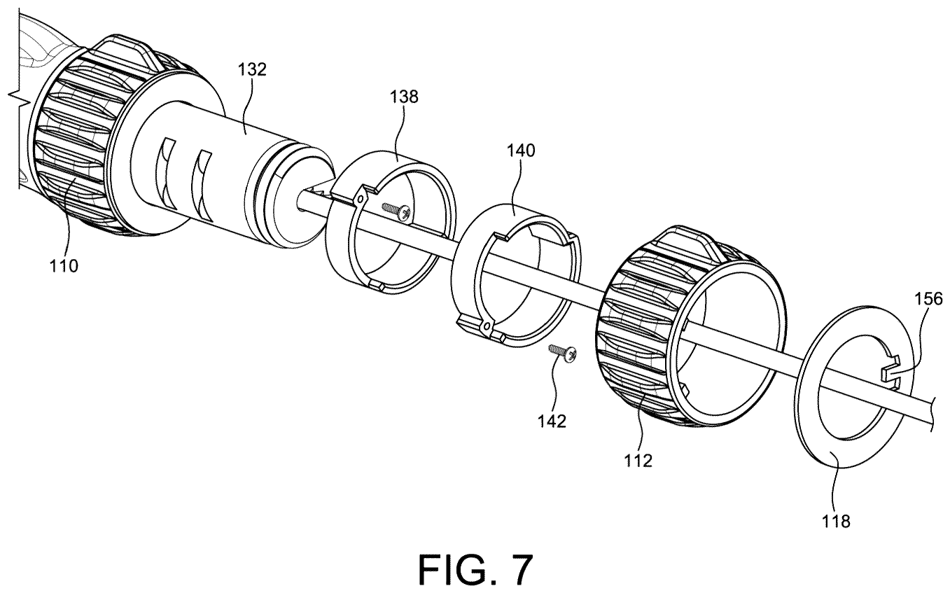

FIG. 7 is a perspective view of the third wire holder 138, fourth wire holder 140, distal knob 112, and second anti-rotation washer 118 being positioned over the spindle 132. The operation of distal knob 112 and third wire holder 138 and fourth wire holder 140 is identical to the operation of proximal knob 110 and first wire holder 134 and second wire holder 136. For example, when the distal knob 112 is rotated counterclockwise, the outer shoulder 191 of the fourth wire holder 140 is pushed by the fourth raised portion 198 until the first internal shoulder 188 of the fourth wire holder 140 contacts one of the ribs 131. Returning the distal knob 112 to the neutral position allows the fourth wire holder 140 to return to the neutral position. When the distal knob 112 is rotated clockwise, the outer shoulder 191 of the third wire holder 138 is pushed by the third raised portion 196 until the first internal shoulder 188 of the third wire holder 138 contacts one of the ribs 131. Returning the distal knob 112 to the neutral position allows the third wire holder 138 to return to the neutral position. At any rotational positioning of the proximal knob 110 and the distal knob 112, the rotatable lock knob 114 can be actuated to lock the knobs 110, 112 in place.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a channel for receiving the plurality of control wires and a plurality of openings through which a proximal portion of the plurality of control wires extend;
a first knob rotatably coupled to the spindle, for actuating a first wire holder to manipulate a first control wire of the plurality of control wires to produce a change in a deflection of the medical device in a first direction and for actuating a second wire holder to manipulate a second control wire of the plurality of control wires to produce a change in a deflection of the medical device in a second direction opposite the first direction;
a second knob rotatably coupled to the spindle, for actuating a third wire holder to manipulate a third control wire of the plurality of control wires to produce a change in a deflection of the medical device in a third direction and for actuating a fourth wire holder to manipulate a fourth control wire of the plurality of control wires to produce a change in a deflection of the medical device in a fourth direction opposite the third direction; and
a rotatable lock for locking the first knob and the second knob to maintain a desired shape of the medical device.

2. The handle of claim 1, wherein the spindle further includes a threaded distal end and a longitudinal groove opening to the channel.

3. The handle of claim 2, wherein the spindle includes a pair of ribs extending along the longitudinal groove.

4. The handle of claim 2, further comprising at least one anti-rotation washer, wherein the anti-rotation washer includes a tang configured to be received in the longitudinal groove.

5. The handle of claim 4, wherein the at least one anti-rotation washer includes a first anti-rotation washer positioned between the first knob and the second knob and a second anti-rotation washer positioned between the second knob and the rotatable lock.

6. The handle of claim 1, wherein the rotatable lock includes an inner surface having a plurality of ramps that interact with a cam washer having a plurality of ramps, and wherein rotation of the rotatable lock causes a compressive force that locks the first knob and the second knob.

7. The handle of claim 2, further comprising a nose cap including internal threads configured for mating with the threaded distal end of the spindle.

8. The handle of claim 1, wherein the first knob includes an internal surface having a first raised portion and a second raised portion extending parallel to a longitudinal axis of the handle, and the second knob includes an internal surface having a third raised portion and a fourth raised portion extending parallel to the longitudinal axis of the handle.

9. The handle of claim 3, wherein the first wire holder, second wire holder, third wire holder, and fourth wire holder include a first portion having a first diameter and a second portion having a second diameter.

10. The handle of claim 9, wherein the first wire holder, second wire holder, third wire holder, and fourth wire holder include a first internal shoulder and second internal shoulder located on an inner surface adjacent junctions between the first portion and the second portion.

11. The handle of claim 10, wherein the first wire holder, second wire holder, third wire holder, and fourth wire holder include an outer shoulder located on an outer surface adjacent a junction between the first portion and the second portion.

12. The handle of claim 11, wherein the outer shoulder of the first wire holder is configured to engage with the first raised portion when the first knob is rotated in a first direction and the outer shoulder of the second wire holder is configured to engage with the second raised portion when the first knob is rotated in a second direction.

13. The handle of claim 12, wherein the outer shoulder of the third wire holder is configured to engage with the third raised portion when the second knob is rotated in a first direction and the outer shoulder of the fourth wire holder is configured to engage with the fourth raised portion when the second knob is rotated in a second direction.

14. The handle of claim 10, wherein the first internal shoulder and the second internal shoulder of the first wire holder, second wire holder, third wire holder, or fourth wire holder are configured to contact the pair of ribs to limit rotation of the first wire holder, second wire holder, third wire holder, and fourth wire holder, respectively, between a neutral position and a maximum deflection position.

15. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a threaded distal end, a channel for receiving the plurality of control wires, and a plurality of openings through which a proximal portion of the plurality of control wires extend;
a first knob rotatably coupled to the spindle, for actuating a first wire holder to manipulate a first control wire of the plurality of control wires to produce a change in a deflection of the medical device in a first direction and for actuating a second wire holder to manipulate a second control wire of the plurality of control wires to produce a change in a deflection of the medical device in a second direction opposite the first direction;
a rotatable lock for locking the first knob to maintain a desired shape of the medical device; and
a nose cap including internal threads configured for mating with the threaded distal end of the spindle.

16. The handle of claim 15, further comprising a second knob rotatably coupled to the spindle, for actuating a third wire holder to manipulate a third control wire of the plurality of control wires to produce a change in a deflection of the medical device in a third direction and for actuating a fourth wire holder to manipulate a fourth control wire of the plurality of control wires to produce a change in a deflection of the medical device in a fourth direction opposite the third direction.

17. The handle of claim 15, wherein the nose cap is configured to retain a proximal end of a tubular member in the channel.

18. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a channel for receiving the plurality of control wires and a plurality of openings through which a proximal portion of the plurality of control wires extend, each of the plurality of openings including a plurality of curved surfaces;
a first knob rotatably coupled to the spindle, for actuating a first wire holder to manipulate a first control wire of the plurality of control wires to produce a change in a deflection of the medical device in a first direction and for actuating a second wire holder to manipulate a second control wire of the plurality of control wires to produce a change in a deflection of the medical device in a second direction opposite the first direction;
a second knob rotatably coupled to the spindle, for actuating a third wire holder to manipulate a third control wire of the plurality of control wires to produce a change in a deflection of the medical device in a third direction and for actuating a fourth wire holder to manipulate a fourth control wire of the plurality of control wires to produce a change in a deflection of the medical device in a fourth direction opposite the third direction; and
a rotatable lock for locking the first knob and the second knob to maintain a desired shape of the medical device.

19. The handle of claim 18, wherein the plurality of curved surfaces includes a first curved surface and a second curved surface extending in a plane orthogonal to a longitudinal axis of the handle, and a third curved surface extending in a plane parallel to the longitudinal axis of the handle.

20. The handle of claim 19, wherein the first curved surface and the second curved surface are configured such that the plurality of openings are larger at an exterior surface of the spindle and smaller at the channel.
